(19) United States
(12) Patent Application Publication
(10) Pub. No.: US 2022/0061698 A1
(43) Pub. Date: Mar. 3, 2022

(54) GAIT ANALYSIS DATA TREATMENT

(71) Applicant: LUXEMBOURG INSTITUTE OF SCHIENCE AND TECHNOLOGY (LIST), Esch-sur-Alzette (LU)

(72) Inventor: Foued MELAKESSOU, Russange (FR)

(21) Appl. No.: 17/414,776

(22) PCT Filed: Dec. 17, 2019
(86) PCT No.: PCT/EP2019/085672

§ 371 Date: Jun. 16, 2021

(30) Foreign Application Priority Data
Dec. 21, 2018 (LU) LU101071

(51) Int. Cl. A61B 5/103; A61B 5/00

(52) U.S. Cl. CPC A61B 5/1038; A61B 5/6807; A61B 2560/0214; A61B 2562/0247; A61B 5/0022

(57) ABSTRACT

A method for analyzing the gait of a user comprising the steps of: providing an article of footwear with a plurality of pressure sensors measuring the pressure applied thereon and generating output signals; providing an evaluation module with a transmitter, the evaluation module receiving the output signals of the pressure sensors; and providing a computing device with a receiver for receiving data transmitted by the evaluation module; the module being configured for: computing stride key indicators (SKI) based on curves; and transmitting the stride key indicators (SKI) to a computing device, and the corresponding system.

The present invention is the US national stage under 35 U.S.C. § 371 of International Application No. PCT/EP2019/085672 which was filed on Dec. 17, 2019, and which claims the priority of application LU 101071 filed on Dec. 21, 2018, the content of which (text, drawings and claims) are incorporated here by reference in its entirety.

### FIELD

The invention is directed to the field of human motion analysis, more particularly to a method and a system for analyzing the gait of a user.

### BACKGROUND

The gait analysis is the systematic study of animal locomotion. The gait analysis is used to assess and treat individuals with conditions affecting their ability to walk. It is also commonly used in sports biomechanics to help athletes run more efficiently and to identify posture-related or movement-related problems in people with injuries. To calculate the kinetics of gait patterns, most labs have floor-mounted load transducers, which measure the ground reaction forces and moments.

In order to simplify the complex installation required for analyzing gait motion, an insole comprising pressure sensors has been developed. This wearable insole solution has been found to be particularly useful. The simplicity of the insole fitted with sensors and the development of smart devices such as smartphone make accessible such a technology to any users. For example, it could benefit the sportsman, people with mobility problem and people with any kind of physical, mobile or dynamic activities such as dancers to have their motions monitored outside a lab.

However, the amount of data recorded for the analysis the gait poses a problem. Ordinary smart devices do[[es]] not have enough memory to record for instance an entire day of activity.

### SUMMARY

The invention has for technical problem to provide a solution to at least one drawback of the above cited prior art. More specifically, the invention has for technical problem to provide a solution to improve the way to store the data related to the gait analysis.

For this purpose, the invention is directed to a method for analyzing the gait of a user comprising the steps of: providing an article of footwear with a plurality of pressure sensors measuring the pressure applied thereon and generating output signals; providing an evaluation module with a transmitter, the evaluation module receiving the output signals of the pressure sensors; and providing a computing device with a receiver for receiving data transmitted by the evaluation module; the module being configured for: recording, during a plurality of stride cycles, the pressure applied to each pressure sensor to obtain a respective curve of pressure as a function of time for each pressure sensor, the respective curve being determined from the output signals; determining an initial time for each of the stride cycles on the basis of the pressure curves; segmenting each curve at each of the initial times; superimposing the segmented curves of at least one sensor; computing stride key indicators based on the superimposed segmented curves; and transmitting the stride key indicators to the computing device.

According to an exemplary embodiment, the evaluation module is integrated into a garment.

According to an exemplary embodiment, the evaluation module is integrated into the article of footwear.

According to an exemplary embodiment, the initial times are common to all the pressure curves and are determined by the time when the pressure and/or the pressure gradient changes from zero to a non-zero value on at least a first particular sensor, for instance the sensor that is positioned at the most rear position of the footwear.

According to an exemplary embodiment, the initial times are common to all the pressure curves and are determined by the time when at least a pressure recorded from the pressure curves changes from zero to a non-zero value and all other pressures remain equal to a zero value.

According to an exemplary embodiment, the module is configured for determining a final time for each of the stride cycles on the basis of the pressure curves, the final times being common to all the pressure curves and being determined by the time when the pressure changes from non-zero to a zero value on at least a second particular sensor, for instance the sensor that is positioned at the most front position of the article of footwear.

According to an exemplary embodiment, the module is configured for determining a final time for each of the stride cycles on the basis of the pressure curves, the final times being common to all the pressure curves and being determined by the time when at least a pressure from the pressure curves changes from a non-zero to a zero value and all other pressures remain equal to a zero value.

According to an exemplary embodiment, the stride key indicators are determined individually for each sensor.

According to an exemplary embodiment, the stride key indicators are based on the curves of pressure of all the sensors, in various instances a weighted sum of the curves of pressure of all the sensors, for example a sum of the curves of pressure of all the sensors.

According to an exemplary embodiment, the stride key indicators are selected from the group consisting of: the maximum pressure over a stride cycle, the average pressure over the stride cycle, the duration of the stride cycle, the duration of the stride cycle, the point in time when the pressure changes from a non-zero value to a zero value, the duration when the pressure curve is not equal to zero, the integral of the pressure over the duration of the stride cycle, a linear combination thereof.

According to an exemplary embodiment, the stride key indicators further comprise the stand duration over a stride cycle.

According to an exemplary embodiment, the position of the center of pressure is calculated at each moment in time based on the curves of pressure of all the sensors.

According to an exemplary embodiment, the stride key indicators are further selected from the group consisting of: the distance travelled by the position of the center of pressure per cycle, the width of the path traveled by the center of pressure, the length of the path covered by the center of pressure, the average center of pressure per cycle, a linear combination thereof.

According to an exemplary embodiment, acceptable values or ranges of values are defined for each stride key indicator and when one of the stride key indicators departs from its acceptable values or ranges of values, a signal is transmitted to the computing device.

According to an exemplary embodiment, the evaluation module is configured to generate a new key indicator based on the detected persistent variations of the curves prior or during the detection of a key indicator departing from its acceptable values or ranges of values.

According to an exemplary embodiment, the evaluation module is configured to detect a change of gait based on the detected persistent variations of the curves prior or during the detection of a key indicator departing from its acceptable values or ranges of values.

According to an exemplary embodiment, the acceptable values or ranges are preset or based on averaged or reference values of previous cycle.

According to an exemplary embodiment, the stride key indicators are determined on a predetermined number of cycles, in various instances 10 cycles or a temporal window, for example amounting to 30 seconds.

According to an exemplary embodiment, the stride key indicators are determined on a variable number of cycles, the variable number being dependent on the detected persistent variations of the curves prior or during the detection of a key indicator departing from its acceptable values or ranges of values.

According to an exemplary embodiment, the recorded data are overwritten after the number of cycles is reached.

According to an exemplary embodiment, the evaluation module comprises a memory, a battery and a switching device, the switching device being configured for determining a remaining capacity of the memory and/or a level of charge of the battery; upon detection of the remaining capacity of the memory and/or the level of charge of the battery being below a predetermined threshold, a plurality of pressure curves of other stride cycles is transmitted to the computing device.

According to an exemplary embodiment, the computing device upon receiving the plurality of pressure curves of other stride cycles is configured for: recording the pressure applied to each pressure sensor to obtain a respective curve of pressure as a function of time for each pressure sensor, the respective curve being determined from the output signals; determining an initial time for each of the stride cycles on the basis of the curves; segmenting each curve at each of the initial times; superimposing the segmented curves of at least one sensor; computing the stride key indicators based on the superimposed segmented curves.

According to an exemplary embodiment, a server is provided and the computing device is configured to transfer the data corresponding to the plurality of pressure curves of other stride cycles to the server.

According to an exemplary embodiment, the computing device is a smart phone.

According to an exemplary embodiment, the pressure sensors are distributed over a portion of the article of footwear adapted to face the sole of a foot.

According to an exemplary embodiment, the article of footwear is a shoe, an insole, a sole of shoe or a sock.

According to an exemplary embodiment, the garment is a shirt, a T-shirt, a sock, trousers, shorts, an insole, a sole of shoe or a sock.

According to an exemplary embodiment, the method further comprises the step of providing a further article of footwear with a plurality of further pressure sensors; measuring the pressure applied thereon and generating output signals, the further article of footwear being a shoe, an insole, a sock or a shoe sole; the module being configured for: recording, during a plurality of further stride cycles, the pressure applied to each of further sensors to obtain a respective curve of pressure as a function of time for each of further pressure sensors, the respective curve being determined from the output signals of the plurality of further pressure sensors; determining an initial time for each of the further stride cycles on the basis of the pressure curves; segmenting each curve at each of the initial times; superimposing the segmented curves of at least one further sensor; computing stride key indicators based on the superimposed segmented curves; and transmitting the stride key indicators to the computing device.

According to an exemplary embodiment, the evaluation module is configured for computing step key indicators based on at least of the plurality of stride cycles and the plurality of further stride cycles.

According to an exemplary embodiment, the evaluation module is configured for computing step key indicators based on at least of the plurality of stride cycles, the plurality of other stride cycles and the plurality of further stride cycles.

The invention is also directed to a system for analyzing the gait of a user comprising: an article of footwear with a plurality of pressure sensors measuring the pressure applied thereon and generating output signals; an evaluation module with a transmitter, the evaluation module receiving the output signals of the pressure sensors; and a computing device with a receiver for receiving data transmitted by the evaluation module; the evaluation module being configured for: recording, during a plurality of stride cycles, the pressure applied to each pressure sensor to obtain a respective curve of pressure as a function of time for each sensor, the respective curve being determined from the output signals; determining an initial time for each of the stride cycles on the basis of the curves; segmenting each curve at each of the initial times; superimposing the segmented curves of at least one sensor; computing stride key indicators based on the superimposed segmented curves; and transmitting stride the key indicators to the computing device.

According to an exemplary embodiment, the evaluation module is integrated into a garment.

According to an exemplary embodiment, the evaluation module is integrated into the article of footwear.

According to an exemplary embodiment, the article of footwear comprises eight pressure sensors distributed over a portion of the article of footwear facing the sole of a foot.

According to an exemplary embodiment, the pressure sensors are adapted to measure a pressure between 0.1 and 7 bars, in various instances between 0.1 and 6 bars.

According to an exemplary embodiment, the article of footwear is a shoe, an insole, a sole of shoe or a sock.

According to an exemplary embodiment, the garment is a shirt, a T-shirt, trousers, shorts, an insole, a sole of shoe or a sock.

The invention is interesting in that it provides a way to reduce the energy consumption of the gait evaluation unit by sharing the workload with the computation unit. The invention allows monitoring the gait for medical and sport purpose, wherein a practitioner or a trainer is directly informed by a change in the gait of patient or sportsman, respectively. The invention permits the miniaturization of the electronic components. The invention analyses the data recorded based on a leaning algorithm, improving the quality of the data computed.

### DRAWINGS

Other features and advantages of the present invention will be readily understood from the following detailed description and drawings among them:

FIG. 1 shows a sole with pressure sensors, in accordance with various embodiments of the invention.

FIG. 2 depicts the superposition method applied to a plurality of pressure curves measured, in accordance with various embodiments of the invention.

FIG. 3 represents the sum of a plurality of pressure curves measured, in accordance with various embodiments of the invention.

FIG. 4 describes trajectories of the geometric center of pressure, in accordance with various embodiments of the invention.

FIG. 5 shows an insole combined with a server, in accordance with various embodiments of the invention.

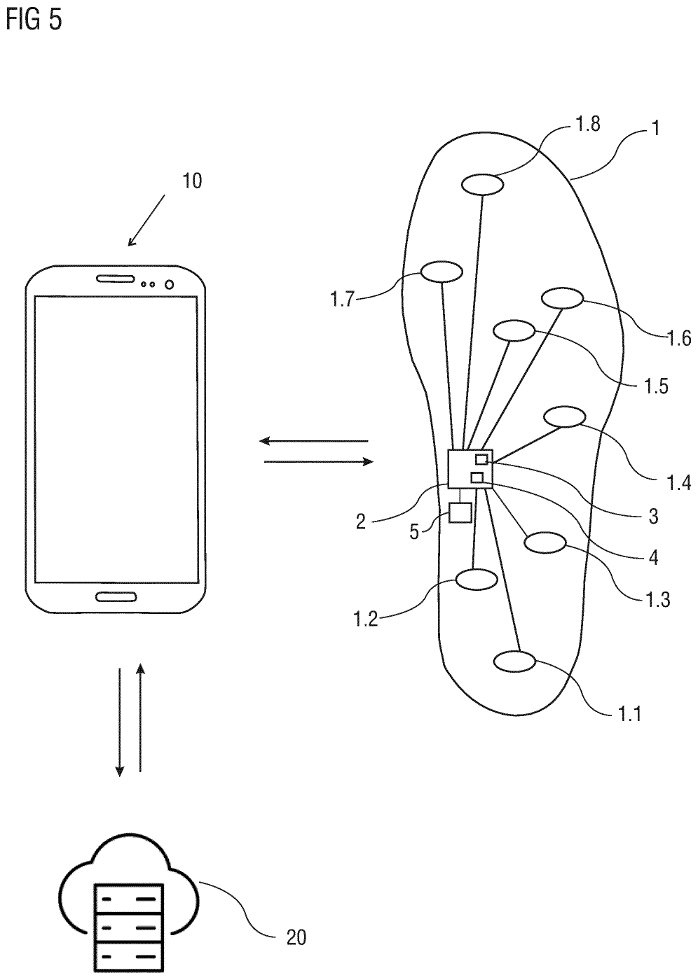

FIG. 6 illustrates two insoles with a wireless connection whit a T-shirt, in accordance with various embodiments of the invention.

### DETAILED DESCRIPTION

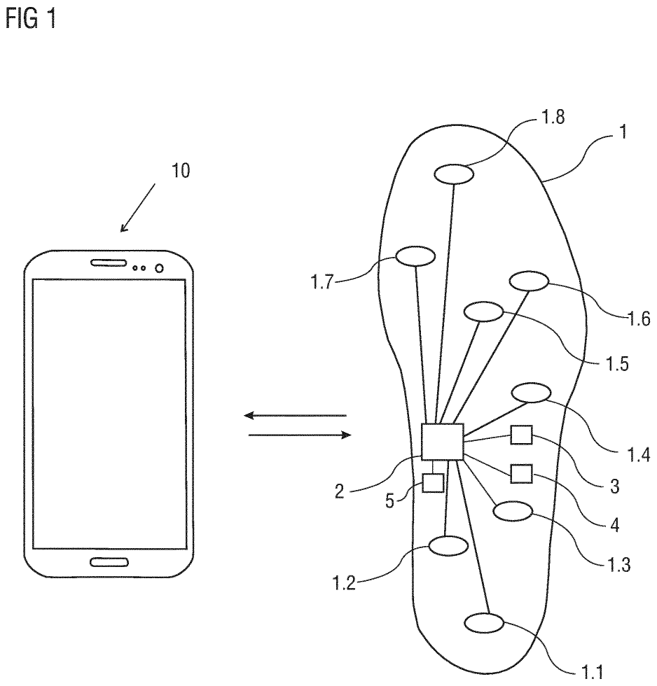

FIG. 1 depicts an article of footwear 1, more precisely an insole 1 equipped with a plurality of pressure sensors 1.1, 1.2, 1.3, 1.4, 1.6, 1.7, 1.8. The invention is not limited to an insole 1. In various embodiments, a plurality of sensors can be provided on a sock, a shoe or a sole of a shoe for instance in order to record the pressure under the sole of a foot. In the present case the insole 1 comprises 8 sensors, but the number of sensors can vary depending on the needs.

The article of footwear 1 according to FIG. 1 comprises an evaluation module 2. In FIG. 1, the transmitter of the evaluation module 2 (data logger) can be connected to an (external) antenna 5 provided in the insole 1. Alternatively, the evaluation module 2 can comprise an internal antenna 5 (not shown). In another alternative, the transmitter can have wire connections to the computing device 10. The evaluation module 2 receives output signals of the plurality of pressure sensors 1.1, 1.2, 1.3, 1.4, 1.6, 1.7, 1.8. The evaluation module 2 is at least configured for recording the pressure applied to each pressure sensor 1.1, 1.2, 1.3, 1.4, 1.6, 1.7, 1.8 to obtain a respective curve of pressure as a function of time. For this exemplary embodiment, the evaluation module 2 is integrated in the insole 1 and connected to the sensors 1.1, 1.2, 1.3, 1.4, 1.6, 1.7, 1.8 via metallic wires. This arrangement allows short electric connections so that the user is not impeded. Alternatively, the evaluation module 2 can be integrated into a garment 8 such as a sock, a shirt, trousers for instance. In various instances, the evaluation module 2 is clipped to a sock or shoe, the evaluation module 2 being connected to the sensors 1.1, 1.2, 1.3, 1.4, 1.6, 1.7, 1.8 of the insole 1 by wires. Alternatively, the communications between the plurality of sensors 1.1, 1.2, 1.3, 1.4, 1.6, 1.7, 1.8 and the evaluation unit 2 can be wireless.

The article of footwear 1 depicted in FIG. 1 also comprises a source of energy 3 such as a battery 3 for the supply of current to the evaluation unit 2 and the plurality of sensors 1.1, 1.2, 1.3, 1.4, 1.6, 1.7, 1.8. In an alternative exemplary embodiment, the battery 3 can be integrated into the evaluation unit 2. The source of energy 3 can be piezo energy using the compression on the insole 1 to generate electricity for the evaluation unit 2 and the plurality of sensors 1.1, 1.2, 1.3, 1.4, 1.6, 1.7, 1.8. Furthermore, the plurality of sensors 1.1, 1.2, 1.3, 1.4, 1.6, 1.7, 1.8 can be also piezo elements generating the electricity needed by the evaluation module 2.

The article of footwear can contain an external memory 4 to increase the memory capacity of the evaluation module 2.

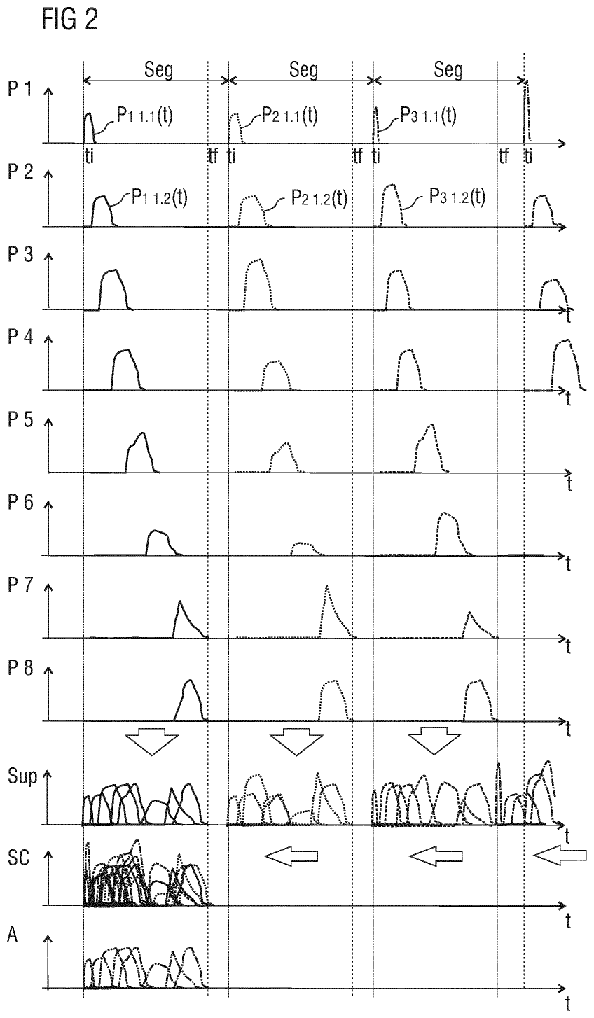

FIG. 2 illustrates the data recorded by the plurality of sensors 1.1, 1.2, 1.3, 1.4, 1.6, 1.7, 1.8 mounted into the article of footwear 1. FIG. 2 shows pressure curves, wherein one graph P1-P8 is displayed for each sensor. The x-axis corresponds to the time, while the y-axis shows the pressure recorded. The pressure curves can be periodic or quasi periodic and show cycles corresponding to the strides. For the quasi periodic cycle, the period can change from one cycle to another. A stride cycle starts, for instance during a walk or run, when a sensor positioned at the rear most position 1.1 of the insole 1 detects the contact of a shoe with the ground and ends when the same sensor 1.1 is pressed at the beginning of the next stride.

A stride cycle according to the invention also encompasses transient phase. For instance, a dancer stepping from the tip a foot activating the most front sensor 1.8 to a position where the dancer steps on a heel activating the most rear sensor 1.1. In this case, two transient cycles take place. In the first one, only the most front sensor 1.8 is activated, while all other sensors remain inactivate. In the second one, the most rear sensor 1.1 is activated, while all other sensors remain inactivate. The definition of a stride can be adapted to the final use, e.g. running, climbing, walking, classical dance.

Stride key indicators are determined by first segmenting the measured curves as shown in FIG. 2, where an initial time ti is determined for each stride cycle. The initial time ti can be detected when the pressure sensor 1.1 positioned on the rear most position change from a non-zero value to a zero value. The initial time ti for a stride can be defined as being the same for all pressures recorded as shown in FIG. 2. A segment Seg can be extracted for a given pressure curve. The segment Seg can start at the initial time ti and can end at the beginning of the initial time ti of the next stride cycle. This operation can be repeated for all the other output values, corresponding to the remaining N−1 segments Seg, N being the number of pressure sensors. All segments Seg can then be superposed on each other, as shown in FIG. 2 in graph Sup. The computation of the stride key indicators of the gait based on the superposed segmented curves serves as basis for the stride key indicator SKI, which can be calculated for each stride. In order to reduce the fluctuation from one cycle to another, a collection of pressure curves can be grouped and then averaged over several stride cycles as shown in FIG. 2 in graphs SC and A, respectively.

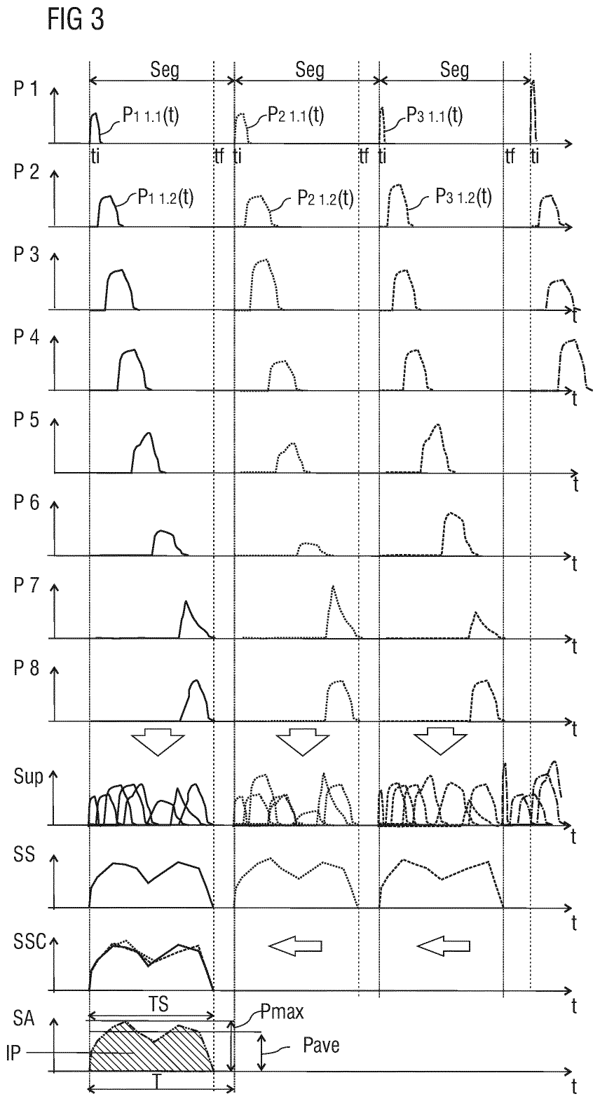

In an exemplary embodiment, as shown in FIG. 3, the plurality of pressure curves can be summed, grouped, and optionally averaged as shown in the figure on graphs SS, SSC and SA, respectively. The sum of the pressures curves is based on the following formula:

$${P}_{i} ( t ) = {\sum }_{k = 1}^{N} {P}_{i 1 . k} ( t )$$

where:

Pᵢ (t) corresponds to the sum of the superposed pressures curves measured for each sensor for stride cycle i; P_{i 1.k}(t) corresponds to the segmented pressure curve recorded for the pressure sensor 1.k for the stride cycle i; N is the number of sensors.

The stride key indicators SKI are selected from the group consisting of: the maximum pressure Pmax over a stride cycle, the average pressure Pave over the stride cycle, the duration T of the stride cycle, the point in time when the pressure changes from a non-zero value to a zero value, the duration during which the pressure curve is not equal to zero, the integral of the pressure IP over the duration of the stride cycle, as shown in FIG. 3.

The point in time when the pressure changes from non-zero to a zero value on sensor 1.8 at the most front position of the article of footwear 1 can correspond to the final time tf. The duration between the initial time ti and the final time tf is the stance duration TS of the stride cycle. The stance duration TS of a stride cycle can be a further stride key indicator SKI. The segment Seg can alternatively be defined as starting at the initial time ti and ending at the final time ft. Also, the swing duration for a cycle is the difference between the stride cycle duration T and the stance duration TS. The swing duration as well the ratio between the stance duration TS and the swing duration can be used as stride key indicators SKI.

In another exemplary embodiment, a position of the center of pressure is calculated at each moment in time based on a linear combination (weighted sum) of the superposed pressure curves of the plurality of pressure sensors. The position of the center of pressure can be determined with the following formulas:

$${x}_{G i} ( t ) = \frac{{Σ}_{k = 1}^{N} {x}_{1. k} {P}_{i 1. k} ( t )}{{Σ}_{k = 1}^{N} {P}_{i 1. k} ( t )} \\ {y}_{G i} ( t ) = \frac{{Σ}_{k = 1}^{N} {y}_{1. k} {P}_{i 1. k} ( t )}{{Σ}_{k = 1}^{N} {P}_{i 1. k} ( t )}$$

where:

x_{G i} (t) corresponds to the position of the geometric center of pressure G according to the x-axis for stride cycle i; y_{G i} (t) corresponds to the position of the geometric center of pressure G according to the y-axis for stride cycle i; x_{1.k} corresponds to the position of the pressure sensor 1.k according to the x-axis; y_{1.k} corresponds to the position of the pressure sensor 1.k according to the y-axis; P_{i 1.k}(t) corresponds to the segmented pressure curve recorded for the pressure sensor 1.k for the stride cycle i; N is the number of sensors.

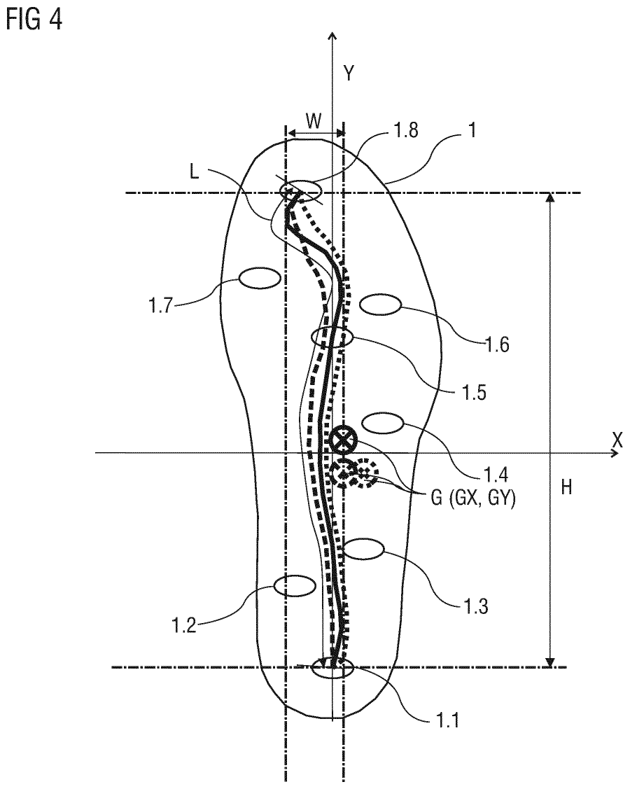

As shown if FIG. 4, the stride key indicators SKI are determined on the basis of the trajectory of the geometric center G selected from the group consisting of: the distance L travelled by the position of the geometric center of pressure G per cycle i, the width W of the path traveled by the center of pressure, the length H of the path covered by the center of pressure. The length H of the path traveled can change when the person just rests on the heel activating a part of the pressure sensors. In FIG. 4, the length and width W are determined for the continuous path line, which corresponds to a stride. This occurs during a transition phase for instance. Also, the average center of pressure is a further key indicator SKI and determined for each cycle with the coordinates (GX, GY), with the following formulas:

$$G X = \frac{{\int }_{ti}^{ti + T} {x}_{G i} ( t ) d t}{T}$$

$$GY = \frac{{\int }_{ti}^{ti + T} {y}_{G i} ( t ) d t}{T}$$

where:

x_{G i} (t) corresponds to the position of the geometric center of pressure G according to the x-axis for stride cycle i; y_{G i} (t) corresponds to the position of the geometric center of pressure G according to the x-axis for stride cycle i; T is the duration of a stride cycle (T can change from one stride cycle to another cycle). The trajectories of the geometric center of pressure can be averaged/smoothed to reduce the noise/fluctuation.

In an exemplary embodiment, the stride key indicators SKI are monitored by the evaluation module 2. For instance, acceptable values or ranges of values are defined for each stride key indicator SKI. When one of the stride key indicators SKI departs from its acceptable values or ranges of values, a signal is transmitted to the computing device 10.

Furthermore, the evaluation module 2 is configured to generate a new key indicator SKI based on the detected persistent variations of the curves prior or during the detection of a stride key indicator SKI departing from its acceptable values or ranges of value. For instance, an evaluation module 2 is mounted on a shoe. In an initial phase, the pressures 1.1, 1.2, 1.3, 1.4, 1.6, 1.7, 1.8 are recorded by insole 1. Then, the user decides to wear a sock equipped with pressure sensors 1.1, 1.2, 1.3, 1.4, 1.5, 1.6, 1.7, 1.8 replacing the insole 1. The transition from one article of footwear to another one implies a change in the coordinates of the pressure sensors. This required an adaptation of the computation of stride key indicators SKI, because the relative distances between the sensors 1.1, 1.2, 1.3, 1.4, 1.5, 1.6, 1.7, 1.8 are altered.

The introduction of the stride key indicators SKI allows a significant reduction in the amount of information, simplifying the management of the memory and reducing the required memory capacity.

Equally, the evaluation module 2 is configured to detect a change of gait based on the detected persistent variations of the curves prior or during the detection of a key indicator departing from its acceptable values or ranges of value. For instance, a user abnormal gait can be detected following an injury of the user. This abnormality can generate an incident that is sent to a practitioner so that further actions can be taken. Equally, the acceptable values or ranges can be preset or be based on averages of previous cycle. For instance, a person climbing a mountain can be monitored. The comparison of the stride key indicators SKI between the beginning of the climb and the end for instance can reflect the tiredness of climber and/or the degree of the slope of the climb. Also, the comparison to the average of previous cycle, allows filtering for slow variations resulting for extrinsic perturbation and not resulting from a change in the gait.

The evaluation module 2 includes an internal memory 4. Optionally, an additional (external) memory 4 is foreseen to increase the memory capacity. The external memory 4 can be interchangeable. The monitoring of the gait generates a large amount of data and therefore needs an appropriate data management. For this purpose, the evaluation module 2 is configured to overwrite previously recorded data after a number of cycles is reached, for instance 10.

In another exemplary embodiment, the computing device 10 can communicate with a remote server 20, which eases the management of the workload of the evaluation module 2. The server 20 can also store a large amount data that can be processed in due time. The server 20 can be configured to compute the key indicators SKI in the same way as the evaluation module 2 or the computing device does. In this configuration, the evaluation module 2 can comprise an internal memory 4 and a battery 3. The workload management is performed by a switching device (not shown) that is part of or external to the evaluation module 2. The switching device is configured for determining a remaining capacity of the memory 4 and/or a level of charge the battery 3. If it is detected that the capacity of the memory 4 and/or the level of charge of the battery 3 is below a predetermined threshold, the plurality of pressure outputs is not recorded in the memory 4 but is transmitted to the computing device 10. Once the computing device 10 receives the data corresponding to the plurality of pressure curves, the computing device 10 determines the stride key indicators SKI for these data. This approach allows discharging the evaluation modules 2. Also, the server 20 can be used to store the data.

The plurality of pressure sensors 1.1, 1.2, 1.3, 1.4, 1.6, 1.7, 1.8 are adapted to measure a pressure between 0.1 and 7 bars, in various instances between 0 and 6 bars. The detection of a passage from non-zero value to a value or form a zero value to a non-zero value could be based on a minimal pressure detection threshold, for example 0.1 bar. The minimal pressure detection threshold can correspond to the resolution of the pressure measurement, therefore amounting to 0.1 bar.

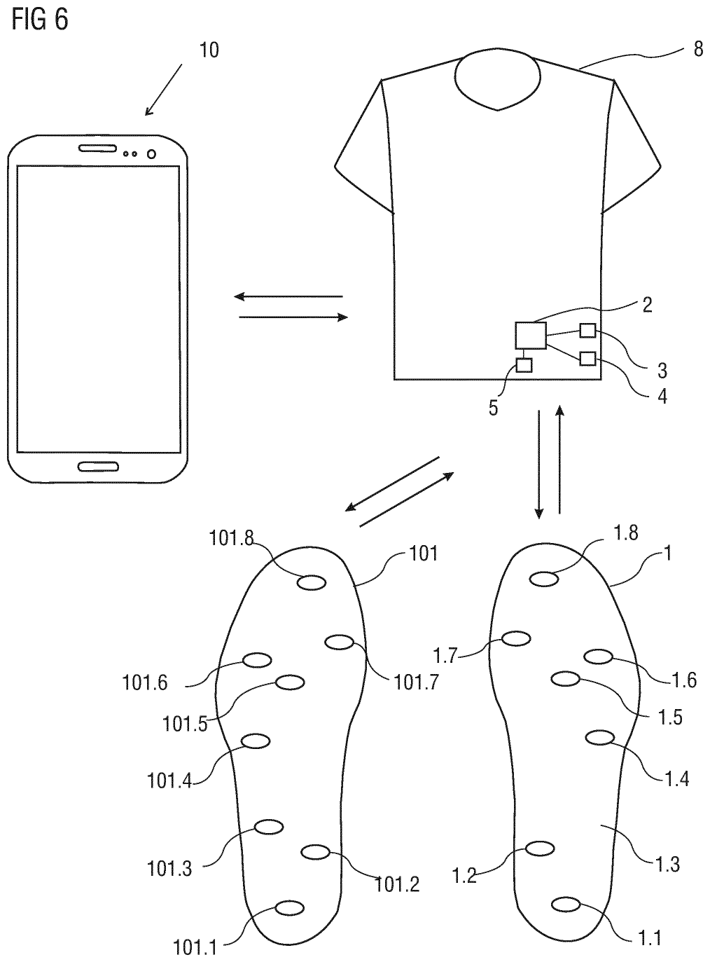

FIG. 6 describes a further exemplary embodiment where the evaluation unit 2 records the pressure data from two separated articles of footwear such as two insoles 1, 101, the right and left respectively. In FIG. 6, the evaluation unit 2 is mounted on a garment 8 (T-shirt). In an alternative the evaluation unit 2 can be mounted on one of the insoles 1,101 so that a pair of interconnected insoles 1, 101 can be sold. Each of the pressure sensors 1.1, 1.2, 1.3, 1.4, 1.6, 1.7, 1.8, 101.1, 101.2, 101.3, 101.4, 101.6, 101.7, 101.8 of the article of footwear 1 and the further article of footwear 101 is provided with a suitable electric supply (not shown) such as a battery, piezo energy source and transmits to the evaluation unit 2 the pressure measurements via a wireless communication (not shown). With such configuration it is possible to determine for instance step key indicators such as the right or left step time.

The stride key indicators SKI can also be treated to represent relative values. For instance, the stand duration TS can be divided by the stride cycle T and the corresponding ratio is transmitted to the computation unit 2. In the case of a pair of insoles, the relative values allow comparing easily the key indicators of the two insoles.

1.-37. (canceled)

38. A meth for analyzing the gait of a user comprising the steps of:
providing an article of footwear with a plurality of pressure sensors measuring the pressure applied thereon and generating output signals;
providing an evaluation module with a transmitter, the evaluation module receiving the output signals of the pressure sensors; and
providing a computing device with a receiver for receiving data transmitted by the evaluation module, the evaluation module being configured for:
recording, during a plurality of stride cycles, the pressure applied to each pressure sensor to obtain a respective curve of pressure as a function of time for each pressure sensor, the respective curve being determined from the output signals;
determining an initial time for each of the stride cycles on the basis of the pressure curves;
segmenting each curve at each of the initial times to obtain segmented curves, each segmented curve corresponding to a stride cycle and to a pressure sensor of the plurality of pressure sensors;
superimposing the segmented curves of at least one pressure sensor of the plurality of pressure sensors to obtain superimposed segmented curves;
computing stride key indicators based on the superimposed segmented curves; and
transmitting the stride key indicators to the computing device.

39. The method according to claim 38, wherein the evaluation module is integrated into one of: a garment, and the article of footwear.

40. The method according to claim 38, wherein the initial times of the plurality of stride cycles are each common to all the pressure curves and are determined by a moment when the pressure changes from zero to a non-zero value on the pressure sensor of the plurality of pressure sensors that is positioned at the most rear position of the footwear.

41. The method according to claim 38, wherein the evaluation module is configured for determining a final time for each of the stride cycles, the final time being determined by the time when the pressure changes from non-zero to a zero value on the pressure sensor of the plurality of pressure sensors that is positioned at the most front position of the article of footwear.

42. The method according to claim 38, wherein a stride key indicator of the strike key indicators is determined individually for each pressure sensor of the plurality of pressure sensors.

43. The method according to claim 38, wherein a stride key indicator of the stride key indicators is determined based on a weighted sum of the curves of pressure of all the pressure sensors.

44. The method according to claim 38, wherein a stride key indicator of the stride key indicators is selected from the group consisting of: the maximum pressure during a stride cycle among all the pressure sensors, the average pressure over the stride cycle, the duration of the stride cycle, the integral of the pressure curve over the duration of the stride cycle, the stance duration, and a linear combination thereof.

45. The method according to claim 38, further comprising calculating at each moment in time a location of the center of pressure based on the curves of pressure of all the sensors.

46. The method according to claim 45, wherein a stride key indicator of the stride key indicators is selected from the group consisting of: a distance travelled by the center of pressure per stride cycle, a width of a path traveled by the center of pressure, a length of a path traveled by the center of pressure, the average location of the center of pressure per stride cycle, and a linear combination thereof.

47. The method according to claim 38, wherein at least one acceptable value or at least one acceptable range of values are defined for each stride key indicator of the plurality of stride key indicators, and wherein when a stride key indicator of the plurality of stride key indicators departs from the corresponding acceptable value(s) or range(s) of values, a signal is transmitted to the computing device.

48. The method according to claim 47, wherein the evaluation module is configured to generate a new key indicator when a stride key indicator of the plurality of stride key indicators has departed from its acceptable value(s) or range(s) of values a pre-defined number of times.

49. The method according to claim 47, wherein an average value is calculated for a given stride key indicator of the plurality of stride key indicators over at least 10 stride cycles, and the acceptable range of values of said given stride key indicator is a range comprising said average value.

50. The method according to claim 38, wherein the evaluation module comprises a memory, a battery and a switching device, the switching device being configured for continuously determining a remaining capacity of the memory and a level of charge the battery, wherein when the remaining capacity of the memory and the level of charge of the battery are above a predetermined threshold, the evaluation module transmits the stride key indicators to the computing device, and when the remaining capacity of the memory or the level of charge of the battery is below a predetermined threshold, the computing device transmits the pressure curves to the computing device.

51. The method according to claim 50, wherein when the remaining capacity of the memory or the level of charge of the battery is below a predetermined threshold, the computing device receives the pressure curves and carries out the following steps in lieu of the evaluation module:
determining an initial time for each of the stride cycles on the basis of the pressure curves;
segmenting each curve at each of the initial times to obtain segmented curves, each segmented curve corresponding to a stride cycle and a sensor;
superimposing the segmented curves of at least one pressure sensor of the plurality of pressure sensors;
computing stride key indicators based on the superimposed segmented curves.

52. The method according to claim 49, wherein the stride key indicators are stored in the memory until they have been transmitted to the computing device and then the memory is erased or newly computed stride key indicators are stored in the memory by overwriting previous stride key indicators.

53. The method according to claim 38, wherein the article of footwear is a first article of footwear, the plurality of pressure sensors is a first plurality of pressure sensor, the plurality of stride cycles is a first plurality of stride cycles, the segmented curves are first segmented curves, the superimposed segmented curves are first superimposed segmented curves, and the method comprises the step of providing a second article of footwear with a second plurality of pressure sensors; the evaluation module being configured for:
recording, during a second plurality of stride cycles, the pressure applied to each of the pressure sensors of the second plurality of sensors to obtain a respective second pressure curve as a function of time for each of the pressure sensors of the second plurality of sensors;
determining a plurality of second initial times, respectively for each stride cycles of the second stride cycles on the basis of the second pressure curves;
segmenting each second pressure curve at each of the second initial times to obtain second segmented curves, each second segmented curve corresponding to a stride cycle of the second plurality of stride cycles and to a sensor of the second plurality of sensors;
superimposing the second segmented curves of at least one sensor of the second plurality of sensors to obtain second superimposed segmented curves; and
computing stride key indicators based on the second superimposed segmented curves; and
transmitting the stride key indicators to the computing device.

54. The method according to claim 53, wherein the evaluation module is configured for computing stride key indicators based on both first and second superimposed segmented curves.

55. A system for analyzing the gait of a user comprising:
an article of footwear with a plurality of pressure sensors measuring the pressure applied thereon and generating output signals;
an evaluation module with a transmitter, the evaluation module receiving the output signals of the pressure sensors; and
a computing device with a receiver for receiving data transmitted by the evaluation module; the evaluation module being configured for:
recording, during a plurality of stride cycles, the pressure applied to each pressure sensor to obtain a respective curve of pressure as a function of time for each sensor, the respective curve being determined from the output signals;
determining an initial time for each of the stride cycles on the basis of the curves;
segmenting each curve at each of the initial times to obtain segmented curves, each segmented curve corresponding to a stride cycle and a sensor;
superimposing the segmented curves of at least one sensor of the plurality of pressure sensors;
computing stride key indicators based on the superimposed segmented curves; and
transmitting stride key indicators to the computing device.

56. The system according to claim 55, wherein the evaluation module is integrated into the article of footwear and the computing device is a smartphone wirelessly connected to the evaluation module.

57. The system according to claim 55, wherein the plurality of sensors is made of at least eight pressure sensors distributed over a portion of the article of footwear and intended to face a sole of a foot, and wherein the pressure sensors are adapted to measure a pressure comprised between 0.1 and 7 bars.
